(19) United States
(12) Patent Application Publication
(10) Pub. No.: US 2014/0207112 A1
(43) Pub. Date: Jul. 24, 2014

(54) HYGIENE BOTTLE

(71) Applicant: Rosemarie Hale, Lansing, MI (US)

(72) Inventor: Rosemarie Hale, Lansing, MI (US)

(21) Appl. No.: 14/155,823

(22) Filed: Jan. 15, 2014

Related U.S. Application Data
(60) Provisional application No. 61/754,013, filed on Jan. 18, 2013.

(51) Int. Cl. A61M 3/02

(52) U.S. Cl. CPC A61M 3/0262
USPC 604/515; 604/216; 604/514

(57) ABSTRACT

A collapsible squeeze bottle suitable for use as a portable personal hygienic washing system provides vigorous cleansing action and can attain a compact configuration conducive to discreet concealment. The squeeze bottle includes non-collapsible top and bottom sections, and a central collapsible section having a plurality of pleats, a spout extending from the top section, and a perforated cap at the terminus of the spout.

### CROSS-REFERENCE TO RELATED APPLICATIONS

This application claims the benefit of U.S. Provisional Application No. 61/754,013, filed Jan. 18, 2013, which is incorporated herein by reference.

### FIELD OF THE DISCLOSURE

This disclosure relates to collapsible containers and dispensers, particularly for dispensing cleansing fluids for personal hygiene purposes.

### BACKGROUND OF THE DISCLOSURE

Known portable personal hygiene washing systems have generally been limited to douching devices that are overly conspicuous for use away from home, and wipes that do not provide adequate cleansing action.

### SUMMARY OF THE DISCLOSURE

Disclosed is a collapsible squeeze bottle suitable for use as a portable personal hygienic washing system that provides vigorous cleansing action and which can attain a compact configuration conducive to discreet concealment. The squeeze bottle includes non-collapsible top and bottom sections, and a central collapsible section having a plurality of pleats, a spout extending from the top section, and a perforated cap at the terminus of the spout.

### BRIEF DESCRIPTION OF THE DRAWINGS

FIG. 1 is a view of a collapsible bottle 10 in an expanded and a collapsed configuration.

FIG. 2 is a view of cleaning solution being poured into bottle 10.

FIG. 3 shows the collapsible bottle in a collapsed form and stowed in a handbag.

### DETAILED DESCRIPTION OF THE DISCLOSED EMBODIMENTS

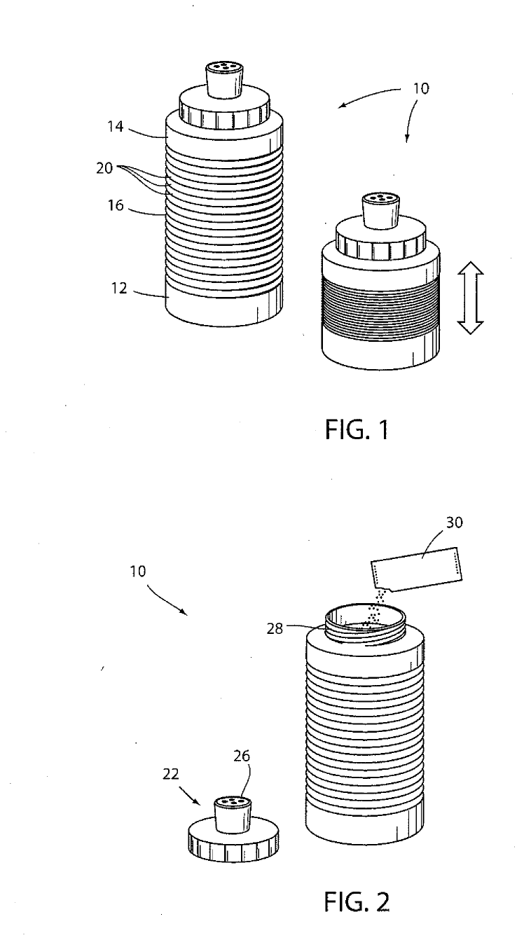

Referring now to FIG. 1, there is shown a view of a collapsible bottle 10 in an expended and a collapsed configuration. The device is comprised of a collapsible bottle that resembles an accordion, and includes a nozzle having multiple openings that is designed for use in the vaginal area. A packet of hygiene concentrate can be added with water to the bottle to form a cleansing solution that can be dispensed in the vaginal area. The device is collapsible into a small, compact package to take on the go. After use, the bottle can be collapsed, stored, and reused at a later time.

Shown in FIG. 1 is a collapsible squeeze bottle 10 having a non-collapsible bottom section 12, a non-collapsible top section 14, a collapsible central section 16 between the top 14 and bottom 12 sections. The collapsible central section 16 has a circumferential side wall comprising a plurality of pleats 20 or folds that allow the central section 16 to be collapsed and expanded as desired or needed. In the fully collapsed state, squeeze bottle 10 is in a compact configuration suitable for storage in a handbag, backpack, briefcase, etc. The pleats 20 provide a bellows like collapsibility and expansibility.

Bottle 10 includes a spout 22 extending from top section 14 and having a terminus 24 at which is located a perforated cap 26. Cap 26 can include one or a plurality of apertures. Spout 22 can be releasably attached to top section 14 by a threaded connection 28, by a snap-type connection or any other suitable connection. Alternatively or additionally, top section 14 can include a primary sub-section 30 that is integral with the collapsible central section (e.g., molded as a single unitary piece) and a lid section 32 that is releasably attached to the primary sub-section by a threaded connection 34, or other suitable connection, such as a snap-type connection.

Bottle 10 can be used in a method of cleansing a portion of a human body by first expanding the bottle (e.g., by pulling the top and bottom sections 12, 14 apart to fully unfold pleats 20). Lid section 32 or spout 22 can be removed to allow the bottle to be filled with a cleansing fluid. The cleansing fluid can be water or a solution of soap and/or a detergent in water. In order to cleanse a body part, the lid 32 and/or spout 22 is replaced and the perforated cap 26 is positioned facing the body part to be cleansed. The bottle is then squeezed such as by moving at least one of the top section 14 and bottom section 12 toward the other of the sections 12, 14 to collapse the bottle, reducing the volume, and increasing the pressure of the fluid in bottle 10 to cause the cleansing fluid to be forcibly ejected from the aperture, or perforated cap 26 and onto the body part to be cleansed. The cleansing fluid can be comprised of a concentrate (solid or liquid) that is dispensed from a packet of pre-measured material that can be diluted with a predetermined amount of water for use as a cleansing fluid.

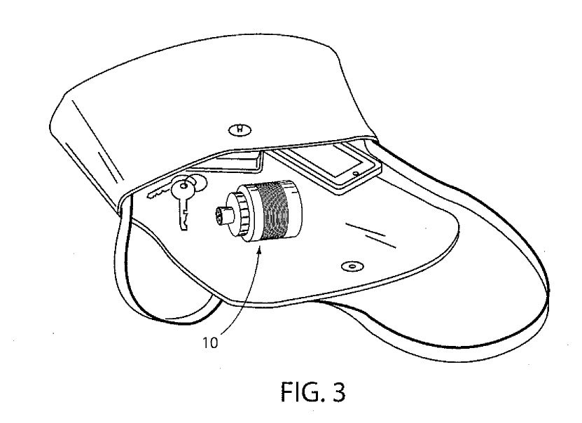

Referring to FIG. 2, there is shown a view of cleansing solution being poured into bottle 10. In operation, a user can remove the cap and pour a packet 30 of solution into the container. The user can then fill the bottle with water, causing it to expand. When full of water, the cap can be replaced, and the bottle can be used as needed. The device can then be reused or disposed of after use. In addition to use in the vaginal area, the present invention can be utilized by parents or caregivers when cleaning infants or toddlers and the elderly who experience incontinence.

Referring now to FIG. 3, the device comprises a uniquely designed bottle with bellows that expand and collapse, similar to an accordion. The device can be stored in a collapsed position, taking up a minimal amount of space. The bottle can be made of pliable plastic or another suitable material. The bottle 10 includes a screw-on or snap-on nozzle cap with a plurality of small holes on the top for external cleansing and direct external contact. The device is offered with a special cleansing solution packet that can be mixed with water. In this way, the present invention enables women to clean themselves or freshen up to prevent embarrassing body odors, discharge, and sweat.

The products general usage is for hygiene purposes, primarily away from home. The applicator comprises a uniquely designed pliable bottle that resembles an accordion. It has a screw on nozzle or cap with multiple openings that is for external cleansing in the genital area. In its collapsed state the applicator is compact, taking up a minimal amount of space. It can be easily stowed in a purse, briefcase, diaper bag or one's pocket. In the expanded configuration, the applicator is filled with water (e.g., 6 ounces). In the privacy of a bathroom stall, squeezing the applicator to direct a spray toward the genital area provides a cleansing and refreshing feeling. Parents can utilize the product for infants and toddlers by laying the child on a changing table.

The applicator can be used by or for an adult male or female, especially an elderly person experiencing incontinence, and women needing a refreshing pick me up during monthly periods. Parents having infants or toddlers in the potty training stage will find the applicator useful away from home. In the medical field caregivers, hospitals and doctor's offices will find the product useful when caring for their patients. The device can be conveniently used in an office, shopping mall or grocery store.

The foregoing descriptions of specific embodiments of the collapsible bottle have been presented for purposes of illustration and description. They are not intended to be exhaustive or to limit the claims to the precise forms disclosed, and obviously many modifications and variations are possible in light of the above teaching. The exemplary embodiment was chosen and described in order to best explain the principles of the disclosed device and its practical application.

What is claimed is:

1. A collapsible squeeze bottle comprising:
(a) a non-collapsible bottom section;
(b) a non-collapsible top section;
(c) a collapsible central section between the non-collapsible bottom section and non-collapsible top section, the collapsible central section having a pleated circumferential side wall that can be at least partially folded from an expanded state to reduce a volume of the squeeze bottle and at least partially unfolded from a compact state to increase the volume of the squeeze bottle;
(d) a spout extending from the top section of the squeeze bottle and having a distal terminus; and
(e) a perforated cap at the terminus of the spout.

2. The bottle of claim 1, wherein the spout is releasably attached to the non-collapsible top section.

3. The bottle of claim 2, wherein the spout is releasably attached to the non-collapsible top section by a threaded connection.

4. The bottle of claim 1, wherein the top section comprises a primary sub-section that is integral with the collapsible central section and a lid section that is releasably attached to the primary sub-section.

5. The bottle of claim 1, wherein the top section comprises a primary sub-section that is integral with the collapsible central section and a lid section that is releasably attached to the primary sub-section by a threaded connection.

6. A method of cleansing a portion of a human body, comprising:
(a) placing a collapsible squeeze bottle of claim 1 into an expanded state;
(b) adding a cleansing fluid to the volume of the collapsible squeeze bottle while it is in the expanded state;
(c) positioning the terminus of the spout adjacent the portion of the human body that is to be cleansed, with the perforated cap facing the portion of the human body that is to be cleansed; and
(d) moving at least one of the non-collapsible bottom section and the non-collapsible top section toward the other of the non-collapsible top section and the non-collapsible bottom section to collapse the collapsible squeeze bottle, thereby reducing the volume of the squeeze bottle and applying pressure to the cleansing liquid to forcibly dispense the cleansing liquid from the perforated cap onto the portion of the human body that is to be cleansed.

7. The method of claim 6, wherein the cleansing fluid comprises water.

8. The method of claim 6, wherein the cleansing fluid comprises water and at least one of soap or detergent.

9. The method of claim 6, wherein adding a cleansing fluid comprises adding a solid or liquid concentrate containing a soap or detergent to the volume of the collapsible squeeze bottle, and adding water to the volume of the collapsible squeeze bottle.

10. The method of claim 9, wherein the solid or liquid concentrate is a pre-measured quantity dispensed from a container for use with a predetermined amount of water.

11. The method of claim 6, wherein the portion of the human body that is to be cleansed is a vaginal area.
